(19) United States
(12) Patent Application Publication
(10) Pub. No.: US 2025/0359766 A1
(43) Pub. Date: Nov. 27, 2025

(54) HEALTH CARE MONITORING AND SMART HOME CONVERGENCE SYSTEM BASED ON BUILT-IN CEILING IOT RADAR SENSOR

(71) Applicants: POSCO E & C Co., Ltd., Pohang-si, Gyeongsangbuk-do (KR); POSCO DX Company Ltd., Pohang-si, Gyeongsangbuk-do (KR); Cheil Electric Co., Ltd., Busan (KR)

(72) Inventors: Hyun Gone Wang, Incheon (KR); Jun Ho Kim, Yongin-si (KR); Su Yeon Jo, Seongnam-si (KR); Jong Hwa Lee, Seoul (KR); Byung Kwon Jeon, Busan (KR); Byeong Hoon Lee, Gimhae-si (KR); Lee Rang Lim, Busan (KR); Seung Hyun Lee, Busan (KR)

(21) Appl. No.: 19/213,930

(22) Filed: May 20, 2025

(30) Foreign Application Priority Data
May 22, 2024 (KR) 10-2024-0066730

(51) Int. Cl. A61B 5/0205; A61B 5/00; A61B 5/024; A61B 5/08; A61B 5/11; A61B 5/113; G16H 50/30

(52) U.S. Cl. CPC A61B 5/0205; A61B 5/024; A61B 5/0816; A61B 5/1117; A61B 5/1126; A61B 5/113; A61B 5/4082; A61B 5/4088; A61B 5/4812; A61B 5/6889; A61B 5/7264; A61B 5/7275; A61B 5/742; G16H 50/30; A61B 2560/0214

(57) ABSTRACT

Proposed is a health care monitoring and smart home convergence system based on a built-in ceiling IoT radar sensor. The health care monitoring and smart home convergence system includes the built-in ceiling IoT radar sensor, and a health analysis part. The built-in ceiling IoT radar sensor is installed into a ceiling of a bedroom, and configured to measure a user's heart rate or respiratory rate on the basis of time of flight (ToF) of a radar signal to generate biosignal data. The health analysis part is configured to analyze the user's sleep pattern on the basis of the biosignal data, and uses the sleep pattern to generate health prediction information on the user's health state.

### CROSS REFERENCE TO RELATED APPLICATION

The present application claims priority to Korean Patent Application No. 10-2024-0066730, filed May 22, 2024, the entire contents of which is incorporated herein for all purposes by this reference.

### BACKGROUND OF THE INVENTION

### Field of the Invention

The present disclosure relates to a smart home convergence system capable of monitoring a user's health state from biosignals, such as heart rate or respiratory rate, measured using an Internet of things (IoT) radar sensor built into a ceiling, and capable of controlling an in-home environment based on IoT.

### Description of the Related Art

The pace of aging in Korea is rapidly increasing, with the country expected to enter an ultra-elderly society by 2025. The number of elderly people aged 65 and over is expected to reach 10 million in 2025, five years from now, and 15 million or more in 2036. The proportion of the elderly population is expected to increase from 16.1% in 2020 to over 20% in 2025 and over 30% in 2035.

In addition, the number of single-person households is on the rise, and according to the Ministry of the Interior and Safety, the number of elderly people living alone increased by 30.9% from 1,253,316 in 2016 to 1,670,416 in 2021. In particular, nearly half (43%) of the lonely deaths that occurred from 2016 to June 2020 were among the elderly aged 65 and over, with 388 (42%) as of June 2020, accounting for more than 40% of lonely deaths each year.

In order to solve these problems, elderly care services are being implemented to provide appropriate care services to vulnerable elderly people who have difficulty managing their daily lives, ensuring a stable retirement life, maintaining their function, health, and preventing deterioration. However, with the increase in the number of people receiving care services and the resulting increase in welfare costs, various research and development efforts are underway on IoT-based smart care systems.

The foregoing is intended merely to aid in the understanding of the background of the present disclosure, and is not intended to mean that the present disclosure falls within the purview of the related art that is already known to those skilled in the art.

### SUMMARY OF THE INVENTION

The present disclosure is directed to providing a health care monitoring and smart home convergence system that predicts a disease that a user may develop to a user on the basis of sleep analysis, and enables the user to be diagnosed with the disease in advance.

The present disclosure is directed to providing a health care monitoring and smart home convergence system that remotely measures a user's respiration or heart rate to monitor the user's health state without disturbing his or her sleep.

The present disclosure is directed to providing a health care monitoring and smart home convergence system that removes noise included in a result of measurement measured by a built-in ceiling IoT radar sensor, thereby providing a reliable result of health state monitoring.

The present disclosure is directed to providing a health care monitoring and smart home convergence system including a built-in ceiling IoT radar sensor that is built in the construction of a single-detached house or an apartment and capable of accurately and efficiently measuring a user's biosignals.

The present disclosure is directed to providing a health care monitoring and smart home convergence system capable of automatically controlling an in-home environment by linking a health monitoring result of a user with IoT devices.

According to an embodiment of the present disclosure, there is provided a health care monitoring and smart home convergence system including: a built-in ceiling IoT radar sensor installed into a ceiling of a bedroom in a home, and configured to measure a user's heart rate or respiratory rate on the basis of time of flight (ToF) of a radar signal to generate biosignal data; and a health analysis part configured to analyze the user's sleep pattern on the basis of the biosignal data, and use the sleep pattern to generate health prediction information on the user's health state.

Herein, the IoT radar sensor may be installed semi-recessed by a fixing frame and a spring clip inside an installation hole formed in the ceiling of the bedroom in a built-in manner, wherein the fixing frame may be inserted into the installation hole to support a lower portion of the IoT radar sensor and may have an opening through which the IoT radar sensor is exposed, and the spring clip may be positioned between the installation hole and the fixing frame, and may be configured to fix the fixing frame within the installation hole with elasticity of the spring clip.

Herein, the IoT radar sensor may be powered by being coupled to a wire harness connected to the installation hole from the inside of the ceiling.

Herein, the IoT radar sensor may include a transmitter configured to output the radar signal at set time intervals, and a receiver configured to receive a reflection signal obtained as the radar signal is reflected, wherein a ratio of the number of the transmitter to the number of the receiver included in the IoT radar sensor may be 1:N (herein, N is an integer of 1 or greater), and a plurality of the receivers may be positioned distributed in a preset area within the IoT radar sensor.

Herein, the IoT radar sensor may be configured to obtain a measurement distance value to the user from the ToF of the reflection signal detected by each of the receivers, and obtain distance variation, which is a difference between the measurement distance values changed for the set time interval, and then compare the distance variation to a cumulative average value of the distance variations accumulated for each of the receivers to determine whether the distance variation is noise.

Herein, the IoT radar sensor may be configured to determine that the distance variation is the noise when the distance variation is out of a set error range of the cumulative average value, and generate the biosignal data on the basis of the remaining distance variations excluding the noise.

Herein, the IoT radar sensor may be configured to detect micro-movement of the user's chest from the distance variation to measure the user's heart rate or respiratory rate.

Herein, the health analysis part may further include a sleep analysis part configured to analyze the user's sleep pattern on the basis of the biosignal data, wherein the sleep analysis part may be configured to classify types of sleep of the user according to number-of-times variations in the respiratory rate or the heart rate.

Herein, the sleep analysis part may be configured to classify the types of sleep into at least one selected from a group of deep sleep, REM sleep, and non-sleep, on the basis of a change range of the number-of-times variations.

Herein, the health analysis part may be configured to generate the health prediction information on the basis of a sleep duration variation for at least one selected from a group of the user's total sleep duration, deep sleep duration, REM sleep duration, and non-sleep duration.

Herein, the sleep analysis part may be configured to determine that the user has left when it is measured that the biosignal data is less than a limit value for a set period of time or longer, and process, as noise, the biosignal data measured while the user has left.

Herein, the health care monitoring and smart home convergence system according to an embodiment of the present disclosure may further include: a positioning detection sensor installed in the bedroom, and configured to remotely measure the user's positioning to generate positioning data, wherein the sleep analysis part may be configured to determine that the user has left when the positioning data corresponds to positioning other than sleep positioning within a preset sleep duration, and process, as noise, the biosignal data measured while the user has left.

Herein, the health care monitoring and smart home convergence system according to an embodiment of the present disclosure may further include: a fall detection sensor installed in the user's home, and configured to detect a fall that occurs to the user while walking in a set area, wherein the health analysis part may be configured to detect that the fall has occurred when a positioning change speed of an object recognized by the fall detection sensor is equal to or greater than a set value and a central axis of the object in the shape of a column of which the central axis is perpendicular to the ground makes positioning change horizontal to the ground and remains in changed positioning for a set period of time or longer.

Herein, the health care monitoring and smart home convergence system according to an embodiment of the present disclosure may further include an environment controller configured to control color, illuminance, and on/off operation of lighting in the user's home depending on at least one selected from a group of the user's types of sleep, life response, current time, weather, whether the user has gone out, and whether the user has fallen.

Herein, the health analysis part may be configured to use the biosignal data to generate the user's resting heart rate (RHR), and generate, on the basis of the resting heart rate, the health prediction information including possibility of occurrence of at least one disease among Alzheimer's disease, Parkinson's disease, stroke, diabetes, and myocardial infarction to the user.

Herein, the health analysis part may be configured to receive, from the user, the user's waist circumference or blood pressure measurement value or both, and generate the health prediction information further including the received waist circumference or blood pressure measurement value.

Herein, the health analysis part may be configured to generate a health score by comparing a reference value with at least one selected from a group of the user's heart rate, respiratory rate, sleep duration, and inactivity time, and provide the health score by including the health score in the health prediction information.

Herein, the health analysis part may be configured to transmit a notification message including the health prediction information to a wall pad installed in the user's home or a pre-registered user terminal.

According to an embodiment of the present disclosure, there is provided a health monitoring method using a health care monitoring and smart home convergence system based on a built-in ceiling IoT radar sensor installed in a home, the health monitoring method including: measuring, by using the built-in ceiling IoT radar sensor installed into a ceiling of a bedroom, a user's heart rate or respiratory rate on the basis of time of flight (ToF) of a radar signal to generate biosignal data; and analyzing the user's sleep pattern on the basis of the biosignal data, and using the sleep pattern to generate health prediction information on the user's health state.

According to an embodiment of the present disclosure, a computer program stored on a medium may be implemented to perform the above-described health monitoring method.

Furthermore, the above-described technical solutions to the technical problems are not all of the features of the present disclosure. Various features of the present disclosure and advantages and effects thereof will be more fully understood with reference to the following detailed exemplary embodiments.

According to the health care monitoring and smart home convergence system according to an embodiment of the present disclosure, a disease that a user may develop can be predicted on the basis of sleep analysis, so that the user can be led to be quickly diagnosed with the disease. In addition, the user can be led to improve his or her the user's living habits, such as the user's sleep pattern or activity level, on the basis of a health monitoring result.

According to the health care monitoring and smart home convergence system according to an embodiment of the present disclosure, a built-in ceiling IoT radar sensor that is built in the construction of a single-detached house or an apartment and capable of accurately and efficiently measuring a user's biosignals can be provided. In this case, a disease can be predicted on the basis of a user's respiration or heart rate measured remotely, so that an accurate sleep pattern can be analyzed without disturbing the user's sleep.

According to the health care monitoring and smart home convergence system according to an embodiment of the present disclosure, noise included in a result of measurement measured by the built-in ceiling IoT radar sensor is removed, thereby providing a reliable result of health state monitoring.

However, the effects that can be achieved by the health care monitoring and smart home convergence system according to embodiments of the present disclosure are not limited to those mentioned above, and other effects not mentioned will be apparent to those skilled in the art from the following description.

### BRIEF DESCRIPTION OF THE DRAWINGS

The above and other objectives, features, and other advantages of the present disclosure will be more clearly understood from the following detailed description when taken in conjunction with the accompanying drawings, in which:

FIG. 1 is a schematic diagram illustrating a health care monitoring and smart home convergence system based on a built-in ceiling IoT radar sensor installed in a home, according to an embodiment of the present disclosure;

FIG. 2 is a schematic diagram illustrating a state in which a built-in ceiling IoT radar sensor is installed in the ceiling, according to an embodiment of the present disclosure;

FIG. 3 is a schematic diagram illustrating a method of installing a built-in ceiling IoT radar sensor in the ceiling, according to an embodiment of the present disclosure;

FIG. 4 is an example diagram illustrating a state in which a built-in ceiling IoT radar sensor is installed, according to an embodiment of the present disclosure;

FIGS. 5A, 5B, and 6 are schematic diagrams illustrating a method of installing a built-in ceiling IoT radar sensor in the ceiling, according to another embodiment of the present disclosure;

FIGS. 7A, 7B, and 8 are schematic diagrams illustrating a method of installing a built-in ceiling IoT radar sensor in the ceiling, according to still another embodiment of the present disclosure;

FIG. 9 is a schematic diagram illustrating the operation of a built-in ceiling IoT radar sensor, according to an embodiment of the present disclosure;

FIG. 10 is a schematic diagram illustrating the arrangement of a transmitter and receivers within a built-in ceiling IoT radar sensor, according to an embodiment of the present disclosure;

FIG. 11 is a table illustrating measurement distance values measured by receivers within a built-in ceiling IoT radar sensor, distance variations, and cumulative average values, according to an embodiment of the present disclosure;

FIG. 12 is a schematic diagram illustrating the operation of a positioning detection sensor and a fall detection sensor, according to an embodiment of the present disclosure;

FIGS. 13A and 13B are schematic diagrams illustrating detection of fall of a user using a fall detection sensor, according to an embodiment of the present disclosure;

FIG. 14 is a table illustrating types of sleep classified by a sleep analysis part, according to an embodiment of the present disclosure;

FIG. 15 is an example diagram illustrating health prediction information based on sleep analysis, according to an embodiment of the present disclosure;

FIGS. 16 and 17 are example diagrams illustrating generation of health prediction information on diabetes and dementia by using resting heart rate, according to an embodiment of the present disclosure;

FIG. 18 is an example diagram illustrating generation of health prediction information including a health score indicative of a health state, according to an embodiment of the present disclosure;

FIG. 19 is an example diagram illustrating the operation of an environment controller, according to an embodiment of the present disclosure;

FIG. 20 is a block diagram illustrating a health care monitoring and smart home convergence system based on a built-in ceiling IoT radar sensor, according to an embodiment of the present disclosure; and

FIG. 21 is a flowchart illustrating a health monitoring method according to an embodiment of the present disclosure.

### DETAILED DESCRIPTION OF THE INVENTION

Hereinafter, embodiments disclosed in the present specification will be described in detail with reference to the accompanying drawings. Throughout the drawings, like or similar elements are denoted by the same reference numerals, and a redundant description thereof will be omitted. The terms “module” and “part” for elements used herein are assigned or used interchangeably for ease of description only and are not intended to have distinct meanings or roles by themselves. That is, the term “part” used in the present disclosure means a software element or a hardware element such as an FPGA or an ASIC, and “part” performs specific functions. However, the term “part” is not limited to software or hardware. The term “part” may be formed so as to be in an addressable storage medium, or may be formed so as to operate one or more processors. Thus, for example, the term “part” may include elements, such as software elements, object-oriented software elements, class elements, and task elements, and may include processes, functions, attributes, procedures, subroutines, segments of program code, drivers, firmware, micro code, circuit, data, database, data structures, tables, arrays, and variables. Functions provided in the elements and “parts” may be combined into a smaller number of elements and “parts”, or may be further divided into additional elements and “parts”.

In addition, in describing an embodiment disclosed in the present specification, if it is determined that a detailed description of the known art related to the present disclosure makes the subject matter of the embodiment disclosed in the present specification unclear, the detailed description will be omitted. In addition, the accompanying drawings are only for easy understanding of the embodiment disclosed in the present specification, and do not limit the technical idea disclosed in the present specification. It is to be understood that the present disclosure includes all modifications, equivalents, and substitutions included in the spirit and the scope of the present disclosure.

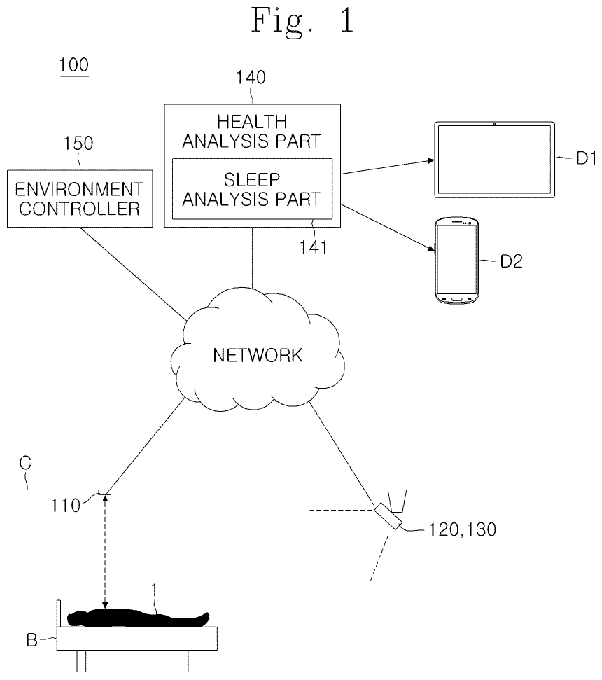

FIG. 1 is a schematic diagram illustrating a health care monitoring and smart home convergence system based on a built-in ceiling IoT radar sensor installed in a home, according to an embodiment of the present disclosure.

Referring to FIG. 1, a health care monitoring and smart home convergence system 100 according to an embodiment of the present disclosure may include an IoT radar sensor 110, a positioning detection sensor 120, a fall detection sensor 130, a health analysis part 140, and an environment controller 150.

Hereinafter, the health care monitoring and smart home convergence system 100 according to an embodiment of the present disclosure will be described with reference to FIG. 1.

A user 1 may be the weak and the elderly, such as an elderly person, an elderly person living alone, and a patient, in addition to a general person living in a house, and the health care monitoring and smart home convergence system 100 may be installed within the user's 1 house.

Herein, examples of the user's 1 house in which the health care monitoring and smart home convergence system 100 is installed may include various types of housing facilities, such as single-detached houses, villas, apartments, and housing for the elderly. According to an embodiment, the health care monitoring and smart home convergence system 100 may be installed in nursing hospitals or nursing facilities for use. The housing for the elderly may be houses built for the elderly or a household including the elderly to live, considering physical and situational characteristics of the elderly.

The user's 1 house in which the health care monitoring and smart home convergence system 100 is installed may include multiple sensors for detecting emergencies, such as falls, fires, and crimes, and may include a wall pad (D1) capable of integrated control of illumination, temperature, humidity in the house on the basis of Internet of things (IoT). Herein, the wall pad (D1) may manage data received from the sensors installed within the house in an integrated manner. According to an embodiment, the health analysis part 130 of the health care monitoring and smart home convergence system 100 may be implemented within the wall pad (D1).

The health care monitoring and smart home convergence system 100 may collect biosignal data, positioning data, and fall data of the user 1 on the basis of the IoT radar sensor 110, the positioning detection sensor 120, and the fall detection sensor 130 installed within the house, and may analyze the user's 1 health state on the basis of the collected data, and may generate health prediction information by predicting a disease that the user 1 may develop.

Herein, the health care monitoring and smart home convergence system 100 may perform communication with the IoT radar sensor 110, the positioning detection sensor 120, and the fall detection sensor 130 over a network. Herein, the communication method is not limited. For example, a communication method using a communication network (for example, a mobile communication network, a wired Internet, a wireless Internet, a broadcast network, and a satellite network) or a short-range wireless communication method between devices may be applied to the network to which the health care monitoring and smart home convergence system 100 and the sensors 110, 120, and 130 are connected.

In the meantime, the health care monitoring and smart home convergence system 100 may generate a report including the health prediction information of the user 1 at regular intervals (daily, weekly, or monthly) or when a particular event occurs (for example, when the analysis indicates an increased risk of disease), and may transmit the generated report in the form of a notification message to the wall pad (D1) or a pre-registered user terminal (D2). Herein, the user terminal D2 may be various terminal devices, such as a mobile communication terminal, a smartphone, a tablet PC, a laptop computer, and a wearable device.

Specifically, the IoT radar sensor 110 of a recessed type may be installed into the ceiling (C) of a bedroom in the home in a built-in manner. The IoT radar sensor 110 may measure the heart rate or the respiratory rate of the user 1 on the basis of time of flight (ToF) of an output radar signal, thereby generating the biosignal data of the user 1. That is, the IoT radar sensor 110 may use ToF of a radar signal to measure a distance to the user 1, and may measure the heart rate and the respiratory rate of the user 1 by detecting the movement of the user's 1 chest on the basis of variations in the measured distance.

Herein, the IoT radar sensor 110 may be installed to be positioned vertically up with respect to the user's 1 breast in order to increase the accuracy of a result of measurement, and may use a radar signal in the millimeter wave frequency band in order to measure changes in the chest in millimeters.

In addition, the position of the user's 1 bed (B) may be determined depending on the position of the IoT radar sensor 110 installed into the ceiling (C), and the user 1 may be guided to lie down so that his or her chest is positioned vertically down from the IoT radar sensor 110 when sleeping. In addition, the user 1 may be guided to take sleep positioning in which the user lies down facing the ceiling.

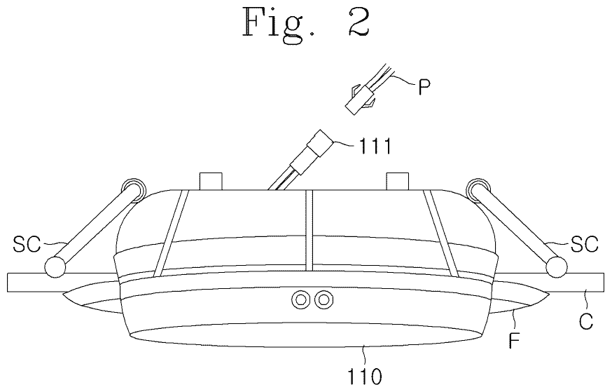

In the meantime, as shown in FIG. 2, the IoT radar sensor 110 may be positioned within an installation hole formed in the ceiling (C) of the bedroom in a built-in manner, and may be installed semi-recessed into the ceiling (C) by a fixing frame (F) and a spring clip (SC) in the installation hole. That is, when the IoT radar sensor 110 of a semi-recessed type is implemented in a built-in ceiling manner, and the IoT radar sensor 110 may be easily installed with the fixing frame (F) and the spring clip (Sc) without the need to install a separate cradle for fixing in the ceiling.

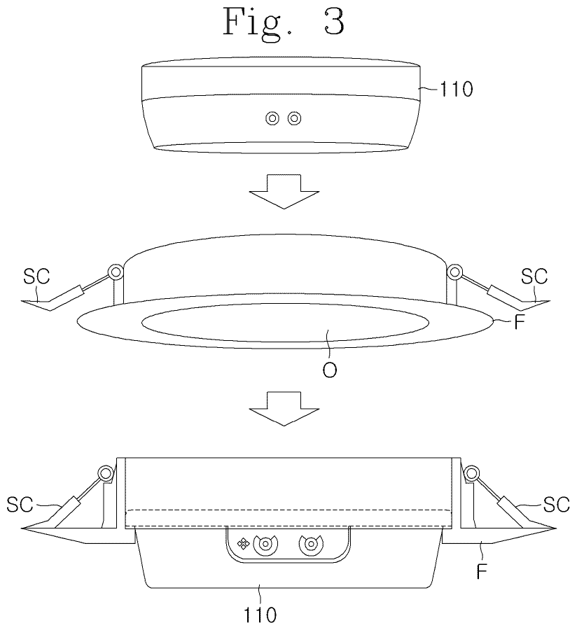

In addition, referring to FIG. 3, the fixing frame (F) may be inserted together with the IoT radar sensor 110 into the installation hole, and the fixing frame (F) may support the lower portion of the IoT radar sensor 110 to prevent the IoT radar sensor 110 from falling out of the installation hole. Herein, an opening (O) is formed in the fixing frame (F), so the IoT radar sensor 110 may be at least partially exposed by the opening (O) and may output, through the opening (O), a radar signal and receive a reflection signal obtained as the radar signal is reflected.

The spring clip (SC) may be positioned between the installation hole and the fixing frame (F), and the spring clip (SC) may apply a pushing pressure between the installation hole and the fixing frame (F) by the elasticity provided by the spring. That is, the fixing frame (F) is pressed between the fixing frame (F) and the installation hole by the spring clip (SC), and is firmly fixed so as not to fall out of the installation hole.

Herein, the IoT radar sensor 110 may be powered by being coupled to a wire harness of a power line (P) connected to the installation hole from the inside of the ceiling. That is, since the hole size for connecting wires is sufficient, as shown in FIG. 2, the wire harness (W) of the IoT radar sensor 110 may be powered by being coupled to the power line (P). Herein, the power line (P) may be implemented in the form of a harness connector. In this way, the wire harness (W) may be used to improve the convenience of installation of the IoT radar sensor 110.

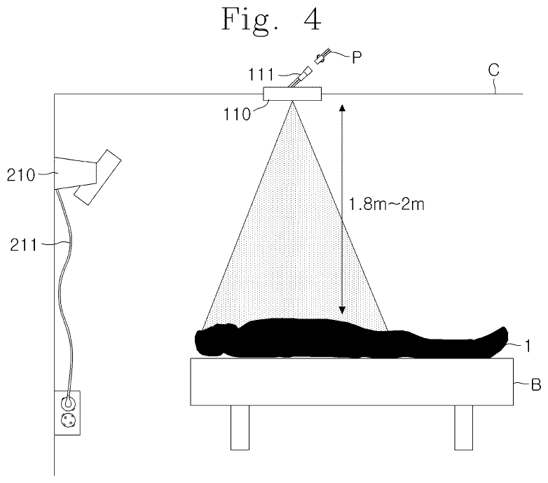

In the meantime, referring to FIG. 4, in the related art, an IoT radar sensor 210 was installed in a form that protrudes from a side of the bedroom, which spoils the appearance of the bedroom and causes difficulties in management, such as cleaning. However, a semi-recessed type of the built-in ceiling IoT radar sensor 110 according to an embodiment of the present disclosure is installed semi-recessed into the ceiling (C), which may provide a neat interior and may be advantageous in terms of management, such as cleaning. Furthermore, regarding the IoT radar sensor 210 in the related art, the power line is exposed when power is connected. However, according to the semi-recessed type of the built-in ceiling IoT radar sensor 110 according to an embodiment of the present disclosure, the power line (P) is connected inside the installation hole, making it easy to supply power without exposure of electrical wiring.

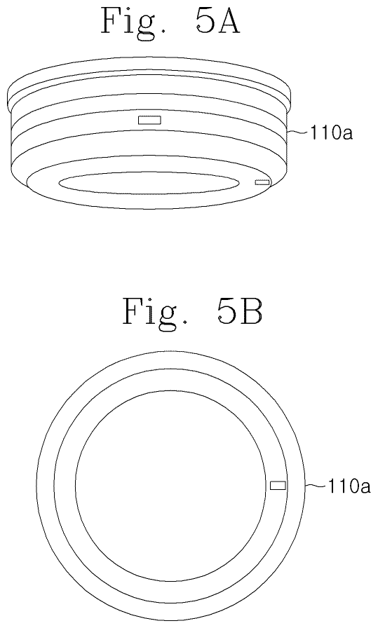

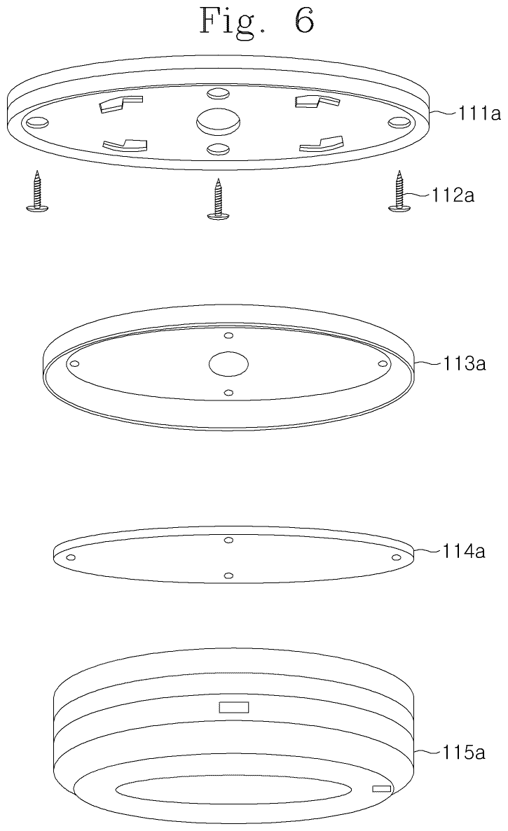

Additionally, the IoT radar sensor 110 may be implemented in various ways according to an embodiment, in addition to a semi-recessed built-in ceiling manner. For example, as shown in FIGS. 5A, 5B, and 6, a ceiling-protruding IoT radar sensor 110a that protrudes from the ceiling may be used. That is, the ceiling-protruding IoT radar sensor 110a may be installed in such a manner that a cradle 111a is fixed to the ceiling with a fixing screw 112a and a sensor lower-casing 113a, a sensor substrate 114a, and a sensor upper-casing 115a are coupled. Herein, in the case of the ceiling-protruding IoT radar sensor 110a, the hole size for connecting wires is formed to be small, which may cause some difficulties in installation. The ceiling-protruding IoT radar sensor 110a may be a fixed type and may have a fixed orientation.

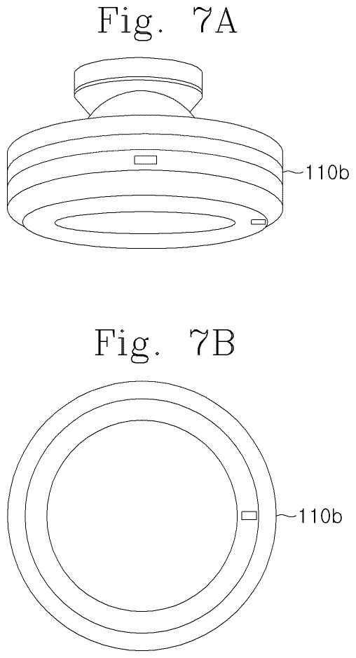

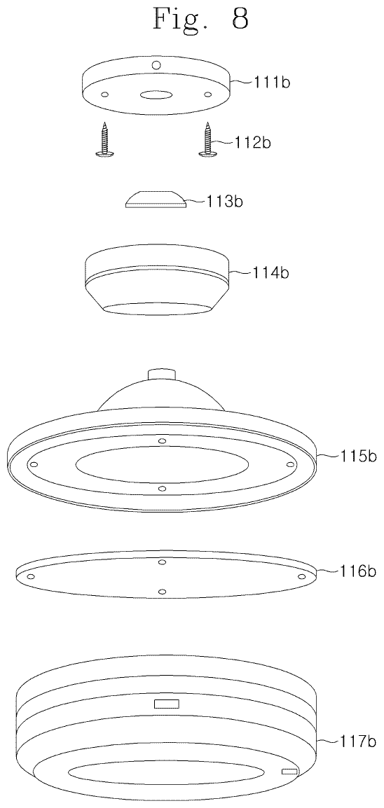

According to still another embodiment, as shown in FIGS. 7A, 7B, and 8, a rotatable IoT radar sensor 110b that protrudes from the ceiling and rotates may be used. That is, a cradle 111b is fixed to the ceiling with a fixing screw 112b, and a support 113b and a rotation holder 114b are installed at the cradle 111b, and then a rotation lower-casing 115b is inserted into the hole in the rotation holder 114b so that the rotation lower-casing 115b is coupled to be rotatable. In addition, the rotatable IoT radar sensor 110b may be installed by coupling a sensor substrate 116b and a sensor upper-casing 117 to the rotation lower-casing 115b. Herein, in the case of the rotatable IoT radar sensor 110b, the hole size for connecting wires is formed to be small, which may cause some difficulties in installation. However, since the rotatable IoT radar sensor 110b is rotatable, the orientation of the rotatable IoT radar sensor 110b may be changed to a desired direction.

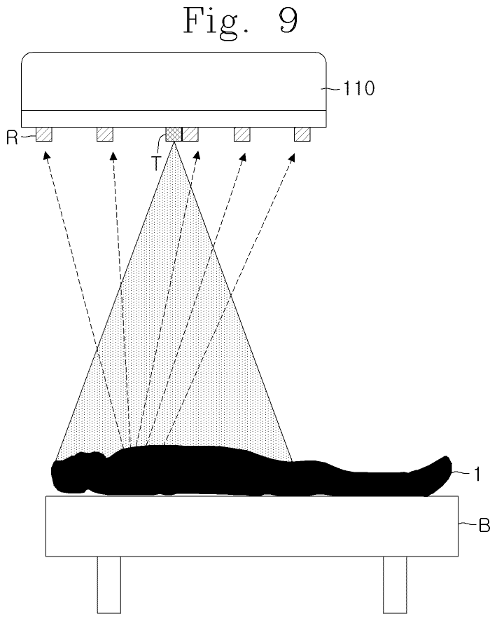

Referring to FIG. 9, the IoT radar sensor 110 may include one transmitter (T) and a plurality of receivers (Rs). The transmitter (T) may output radar signals at set time intervals (for example, 1 msec), and the receivers (Rs) may receive reflection signals obtained as the radar signals are reflected back.

Herein, by measuring ToF of a reflection signal, the distance from the IoT radar sensor 110 to the user 1 may be measured on the basis of the speed of a radar signal. That is, when micro-movement in mm is measured using a radar signal, changes in the user's 1 chest caused by the respiration or heart beat of the user 1 may be detected, thereby measuring the respiratory rate or the heart rate of the user 1. That is, micro-movement of the chest caused by the respiration or the heart beat of the user 1 may be detected from distance variation measured by the IoT radar sensor 110, thereby measuring the heart rate or the respiratory rate of the user 1. For example, the heart rate may be measured by the IoT radar sensor 110 measuring that the boundary of the size of the heart in the chest grows or shrinks as the heart beats.

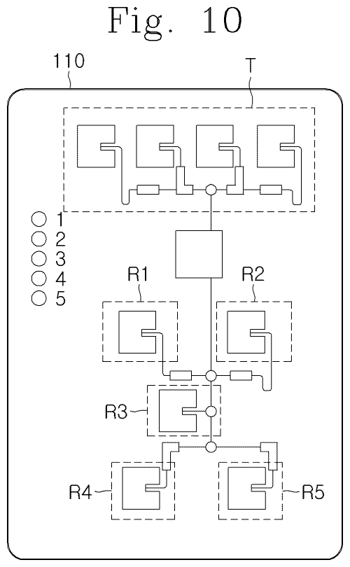

FIGS. 9 and 10 shows the case in which the IoT radar sensor 100 includes one transmitter (T) and a plurality of receivers (Rs). However, an embodiment there are a plurality of transmitters (Ts) is possible. In this case, the ratio of the number of transmitters (Ts) to the number of receivers (Rs) included in the IoT radar sensor 110 may be 1:N (herein, N is an integer of 1 or greater).

When the IoT radar sensor 100 includes a plurality of receivers (Rs), the receivers (Rs) may be positioned distributed in a preset area within the IoT radar sensor 110. As shown in FIG. 10, when the IoT radar sensor 110 includes one transmitter (T) and five receivers (Rs), the receivers (R1, R2, R3, R4, and R5) may be distributed to be positioned at five respective areas: upper left, upper right, center, lower left, and lower right. That is, as shown in FIG. 9, a radar signal may be output to a large area, and a reflection signal resulting from reflection may be divided and input to each of the receivers (R1, R2, R3, R4, and R5). Thus, the respective receivers (R1, R2, R3 R4, and R5) may provide measurement distance values for respective different points. For example, a first receiver (R1) and a second receiver (R2) positioned at the upper left area and the upper right area may provide measurement distance values for the areas from the user's 1 head to shoulders, and a third receiver (R3) positioned at the center area may provide a measurement distance value for the neck and the area above the pit of the stomach, and a fourth receiver (R4) and a fifth receiver (R5) positioned at the lower left area and the lower right area may provide respective measurement distance values for the left and the right side below the pit of the stomach. In this way, the measurement distance values of the respective receivers (R1, R2, R3, R4, and R5) may be matched to the user's 1 body parts, respectively. By applying this, more accurate measurement of the respiratory rate or the heart rate may be achieved.

In the meantime, the measurement distance values measured by the IoT radar sensor 110 may include noise due to a measurement environment, such as a bedroom with inconsistent temperature and humidity, and the user's 1 movement. Therefore, it is necessary to remove noise in order to increase the accuracy of the respiratory rate and the heart rate measured by the IoT radar sensor 110.

Specifically, the IoT radar sensor 110 may obtain a measurement distance value to the user 1 from the ToF of a reflection signal detected by each receiver (R), and may obtain distance variation, which is the difference between measurement distance values changed for a set time interval (for example, 1 msec). That is, the transmitter (T) outputs radar signals at set time intervals, and the distance variation may be obtained by comparing the measurement distance value previously measured and with the measurement distance value currently measured. Afterward, it may be determined whether the current distance variation is noise by comparing the distance variation with the cumulative average value of the distance variations accumulated for each of the receivers (R1, R2, R3, R4, and R5). That is, micro-changes in the chest that may occur due to the heart beat or the respiration are not out of a particular range, so it may be determined whether the current distance variation is noise on the basis of the cumulative average value.

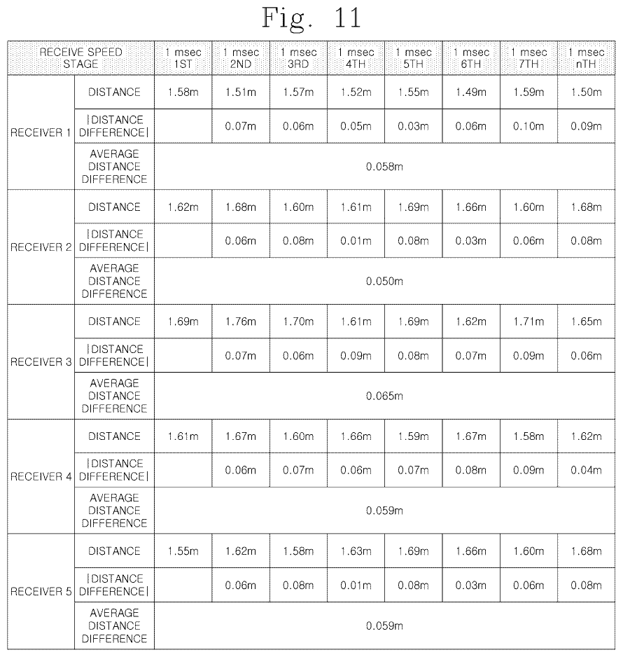

Referring to FIG. 11, in the case of the receiver 1 (R1), the distance measurement value measured at the first time was 1.58 m and the distance measurement value measured at the second time was 1.51 m, so the distance variation was 0.07 m. In addition, the distance measurement value measured at the third time was 1.57 m, so when compared to the distance measurement value of 1.51 m measured at the second time, the distance variation was 0.06 m. In this way, the distance measurement value and the distance variation may be obtained for each of the n times. Herein, it may be calculated that the cumulative average value of the distance variations was 0.058. Therefore, it may be determined whether the distance variation of the distance measurement value measured at the n+1-th time is included within a particular set error range (for example, ±20% or less) of the cumulative average value of 0.058. When the distance variation is out of the set error range, it may be determined that the distance variation is noise. Herein, when the distance variation is not noise, the cumulative average value is updated by adding the distance variation measured at the n+1-th time. However, when it is determined that the distance variation is noise, the noise is not applied and the cumulative average value may remain.

That is, when the distance variations obtained from the reflection signals received from the respective receivers (R1, R2, R3, R4, and R5) are compared to the cumulative average value and within the set error range of ±20% or less, the reflection signals may be regarded as signals reflected back from the actual user 1. Herein, when the distance variation is equal to or greater than a set value, it may be determined that the respiration or the heart beat has been taken once. Herein, different set values may be set for the respiration and the heart beat. In the meantime, when the distance variation obtained from the reflection signal is out of the set error range of the cumulative average value, the distance variation corresponds to noise and may be excluded. The biosignal data may be generated on the basis of the remaining distance variations excluding noise.

The positioning detection sensor 120 may be installed in the bedroom, and may remotely measure the user's 1 positioning to generate positioning data. The fall detection sensor 130 may be installed in the home and may detect the user 1 falling while walking in a set area.

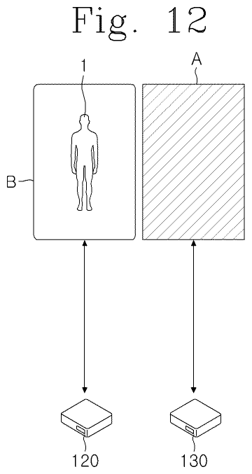

Referring to FIG. 12, the positioning detection sensor 120 may be installed together with the fall detection sensor 130. The positioning detection sensor 120 may be installed facing the bed (B), and may detect the user's 1 positioning on the bed (B). For example, the positioning detection sensor 120 may recognize sleep positioning of the user 1 lying on the bed (B), and positioning of the user sitting on the bed (B), and positioning of the user standing up from the bed (B), distinguishing between each of these positions. Afterward, the positioning detection sensor 120 may generate positioning data representing the user's 1 positioning and may transmit the positioning data to the health analysis part 140. In this case, when the positioning data corresponds to positioning other than sleep positioning within a preset estimated sleep duration, the health analysis part 140 may determine that the user 1 has left and may process, as noise, the biosignal data measured while the user 1 has left.

In addition, the fall detection sensor 130 may be installed facing a preset set area (A) near the bed (B), and may detect the user 1 falling from the bed (B). Herein, the set area (A) is set to be next to the bed (B), but the set area (A) may be set in various positions other than the bedroom. For example, the fall detection sensor 130 may be installed in a place, such as a hallway or a bathroom in the home, where there is a high risk of falling so as to detect the user 1 falling while walking.

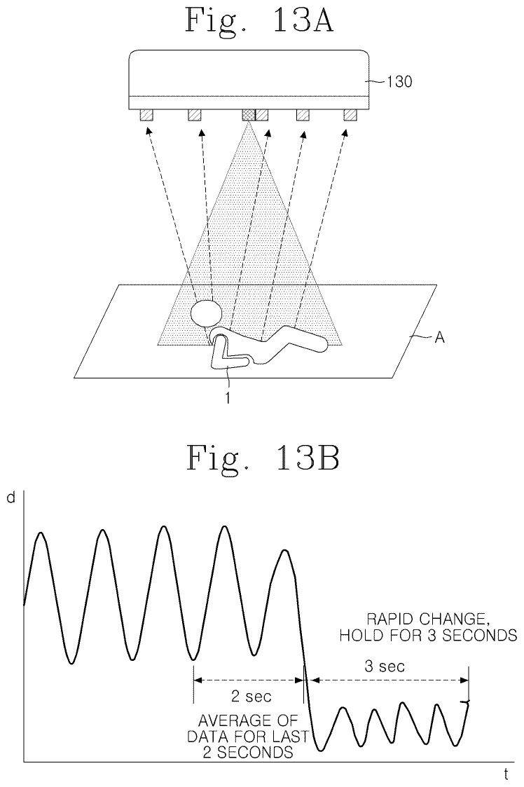

Herein, as shown in FIG. 13A, when the user 1 enters the set area (A), the fall detection sensor 130 may transmit radar signals to the user 1 at regular intervals, and may determine whether the user 1 has fallen on the basis of reflected radar signals. Specifically, when the measurement distance values measured by a plurality of receivers (R1, R2, R3, R4, and R5) of the fall detection sensor 130 changes simultaneously and rapidly, it may be determined that a fall has occurred. For example, as shown in FIG. 13B, when the five receivers measure the distance variation that is different, by 80% or greater, from the cumulative average value of the distance variations for the past 2 seconds in a short period on a per-100 msec basis and this state is maintained for at least 3 seconds, it may be determined that a fall has occurred and fall data may be transmitted to the health analysis part 140. According to an embodiment, after the fall detection sensor 130 recognizes a column-shaped object of which the central axis is perpendicular to the ground within the set area (A), when the positioning change speed of the object is equal to or greater than a set value and the central axis makes positioning change horizontal to the ground, it may be determined that a fall has occurred. Herein, the fall detection sensor 130 directly determines whether a fall has occurred. However, according to an embodiment, the health analysis part 140 may determine whether the user 1 has fallen, on the basis of a result of measurement by the fall detection sensor 130.

The health analysis part 140 may analyze the sleep pattern of the user 1 on the basis of the biosignal data, and may use the sleep pattern to generate the health prediction information on the user's 1 health state. Specifically, the health analysis part 140 may further include a sleep analysis part 141 for analyzing the sleep pattern of the user 1. On the basis of the type of sleep or the quality of sleep analyzed by the sleep analysis part 141, the health analysis part 140 may predict the user's 1 health state and may generate and provide the health prediction information accordingly. Herein, the health analysis part 140 and the sleep analysis part 141 may be implemented on the basis of various types of machine learning models, deep-learning models, and neural network models. According to an embodiment, the health analysis part 140 and the sleep analysis part 141 may be implemented to operate on the basis of a preset rule.

The sleep analysis part 141 analyzes the sleep pattern from biosignal data in a state in which the user 1 is sleeping, so it is necessary to first determine whether the user 1 is sleeping. That is, the sleep analysis part 141 may analyze the user's life pattern based on the collected biosignal data, and may determine whether the user is sleeping or not, on the basis of the user's life pattern. Specifically, the sleep analysis part 141 may use an analysis model based on machine learning or neural network, and may receive the sleep duration, which is the actual time spent sleeping, from the user in order to train the analysis model. In this case, the analysis model may learn the biosignal data collected during the sleep duration to distinguish between the case in which the user is active and the case in which the user is sleeping, and may analyze the sleep pattern on the basis of the biosignal data corresponding to the case in which the user is sleeping.

In the meantime, according to an embodiment, the sleep analysis part 141 may use additional information other than the analysis model for determining whether the user is sleeping, or may use the analysis model and the additional information simultaneously. That is, the additional information, such as the estimated sleep duration, may be provided from the user. In this case, the sleep analysis part 141 may collect biosignal data for the preset estimated sleep duration, and may analyze, on the basis of the biosignal data collected for the estimated sleep duration, the sleep pattern of the biosignal data, such as the respiration or the heart rate during sleeping.

Herein, the estimated sleep duration may be the time that the user 1 specifies in advance as his or her sleep duration. For example, when the user 1 sets his or her estimated sleep duration from 9 p.m. to 4 a.m., the sleep analysis part 141 may collect biosignal data between 9 p.m. to 4 a.m., and may analyze the user's 1 sleep pattern on the basis of the biosignal data.

In addition, according to an embodiment, an illuminance sensor provided in the bedroom may be used to collect illuminance changes in the bedroom as additional information, and the user's 1 estimated sleep duration may be set on the basis of the illuminance changes. Alternatively, the on/off operation of a light in the bedroom may be collected as additional information and the estimated sleep duration may be set. That is, the light in the bedroom may be turned off before the user goes to sleep, and the light in the bedroom may be turned on after the user wakes up, so the time in between may be estimated as the sleep duration. In addition, the charging start time point and the charging end time point of the mobile communication terminal (D2) of the user 1 may be collected as additional information to set the estimated sleep duration. Alternatively, various types of additional information collected with a combination of the above-described methods may be used to set the user's 1 estimated sleep duration.

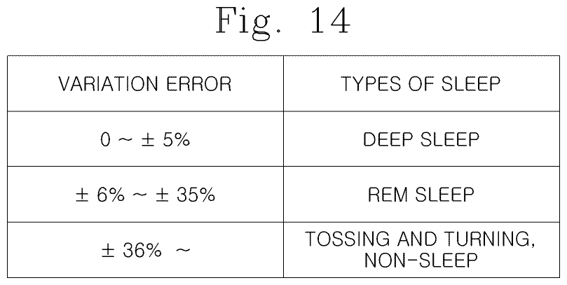

Afterward, the sleep analysis part 141 may classify the types of sleep of the user 1 by using number-of-times variations in the respiratory rate or the heart rate of the user 1. For example, as shown in FIG. 14, the number-of-times variation in the respiratory rate or the heart rate is small, between 0˜ 5%, it may be determined that the user is in a deep sleep state. When the number-of-times variation in the respiratory rate or the heart rate is between 6˜ 35%, the user is in REM sleep. In addition, when the number-of-times variation in the respiratory rate or the heart rate is equal to or greater than 36%, it may be determined that the user is in a non-sleep state in which the user 1 can not fall asleep and tosses and turns. Herein, the number of types of sleep and criteria for classifying the types of sleep may vary depending on an embodiment.

The sleep analysis part 141 may generate sleep data including the types of sleep occurring while the user 1 is sleeping and the time corresponding to each of the types of sleep, and may collect the sleep data at regular time intervals to generate statistics. That is, the sleep analysis part 141 may generate daily sleep data, monthly sleep data, and yearly sleep data for sleep data, and may provide diagrams of trends by sleep type for each period. Afterward, the health analysis part 140 may analyze, on the basis of the sleep data provided by the sleep analysis part 141, change trends for the total sleep duration, the deep sleep duration, the REM sleep duration, and the non-sleep duration to generate the health prediction information of the user 1. That is, the health prediction information may attract the user's 1 interest by providing the user 1 with information on diseases that may occur depending on each sleep duration change, and may also provide the user 1 with information on activities and the amount of required sleep to prevent the diseases.

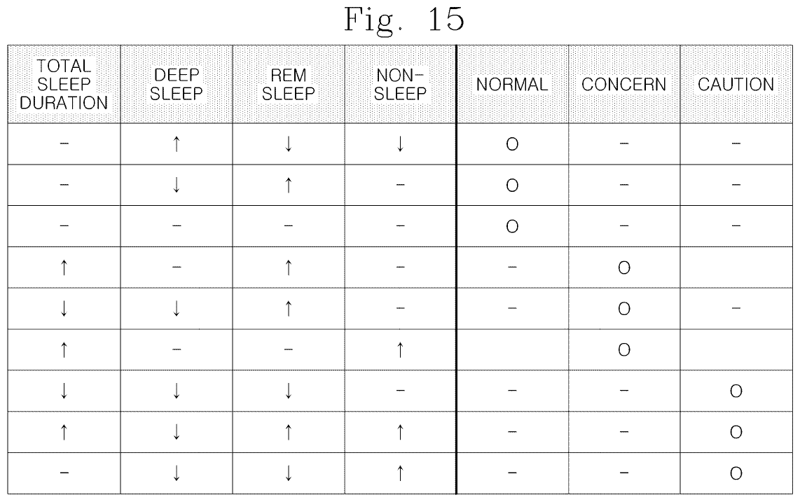

Referring to FIG. 15, the health analysis part 140 may generate the health prediction information on the basis of the change trends for the total sleep duration, the deep sleep duration, the REM sleep duration, and the non-sleep duration, by classifying each of the trends into three states: normal, concern, and caution. However, the present disclosure is not limited thereto, and the health prediction information related to the sleep pattern may be provided to the user 1 in various ways other than the three states.

Specifically, compared to the past sleep patterns, when the total sleep duration remains unchanged, but the REM sleep duration and the non-sleep duration are decreased and the deep sleep duration is increased, the health analysis part 140 may determine that the user 1 has a good sleep pattern and is healthy or normal, and may make classification into “normal”. In addition, compared to the past sleep patterns, when the total sleep duration and the non-sleep duration remain unchanged, but the deep sleep duration is decreased and the REM sleep duration is increased, the health analysis part 140 may also determine that the user 1 has a good sleep pattern and is health or normal, and may make classification into “normal”. In addition, even when the sleep pattern does not change from the sleep pattern for a particular period of time in the past, it may be determined that the sleep pattern is “normal”. That is, a consistent sleep pattern remains continuously, so the health analysis part 140 may determine that there is no problem. However, the extent to which the particular period of time in the past is set may vary depending on the user's 1 state.

In the meantime, when the deep sleep and the non-sleep are the same, but the total sleep duration is increased and the REM sleep is increased, this may be considered normal. However, when only the REM sleep duration is increased, the health analysis part 140 may make classification into “concern” and may persuade the user 1 to be concerned about sleep. In addition, when the total sleep duration is decreased and the deep sleep duration is decreased and the REM sleep duration is increased, the health analysis part 140 may make classification into “concern” and may persuade the user to check the causes of the decrease in the deep sleep duration. When the total sleep duration is increased and the non-sleep is increased as much as the total sleep duration is increased, the health analysis part 140 may make classification into “concern” and may lead the user 1 to fall asleep more easily, for example, increase exercise or drink warm water.

However, when the non-sleep duration is the same and the time required to fall asleep is unchanged, but all the total sleep duration, the deep sleep duration, and the REM sleep duration are decreased, the health analysis part 140 may make classification into “caution” and may inform the user 1 that he or she needs to be careful with his or her health. In addition, the user also needs to be careful when the total sleep duration is increased and the non-sleep duration before falling asleep is increased and the deep sleep duration is decreased and the REM sleep duration is increased. Therefore, the health analysis part 140 may make classification into “caution”. The user also needs to be careful about his or her health when the total sleep duration is unchanged and the deep sleep duration and the REM sleep duration are decreased and the non-sleep duration is increased. Therefore, the health analysis part 140 may generate the health prediction information with classification into “caution”.

Additionally, the health analysis part 140 may use the biosignal data to generate the resting heart rate (RHR) of the user 1, and may generate, on the basis of the resting heart rate, the health prediction information including the possibility of occurrence of various diseases (for example, Alzheimer's disease, Parkinson's disease, stroke, diabetes, and myocardial infarction) to the user 1. Herein, the health prediction information may provide the possibility of occurrence of each disease, being classified into three states: normal, concern, and caution. However, according to an embodiment, the health prediction information may be provided to the user in various ways, for example, making classification into more subdivided states.

Specifically, among the heart rate data measured during sleep, the stable heart rate data during a period with little change in the heart rate data is the resting heart rate. In general, when the resting heart rate increases, the risk of dementia or diabetes may increase. According to an embodiment, the heart rate when the user 1 is in deep sleep may be measured and set as the resting heart rate.

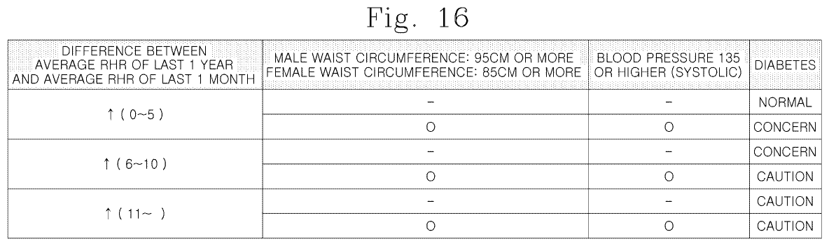

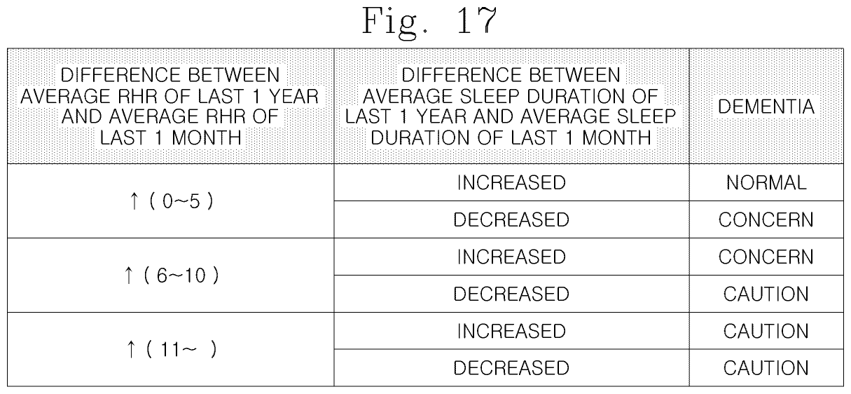

Accordingly, as shown in FIGS. 16 and 17, the health analysis part 140 may provide the health prediction information that classifies, on the basis of the change trends in the resting heart rate, the possibility of occurrence of diabetes or dementia, into “normal”, “concern”, and “caution”. Herein, the health analysis part 140 may increase the reliability of the possibility of occurrence of each disease, further considering the user's 1 waist circumference, blood pressure measurement value, and the change trends for the sleep duration. Herein, the change trends for the sleep duration may be generated from sleep data, but the user's 1 waist circumference or blood pressure measurement value may be separately input from the user 1. In addition, in FIGS. 16 and 17, the average RHR for the last 1 year is a criterion for determination, but this is only an example. In some cases, the average RHR for one week or several months or several years may be a criterion for comparison.

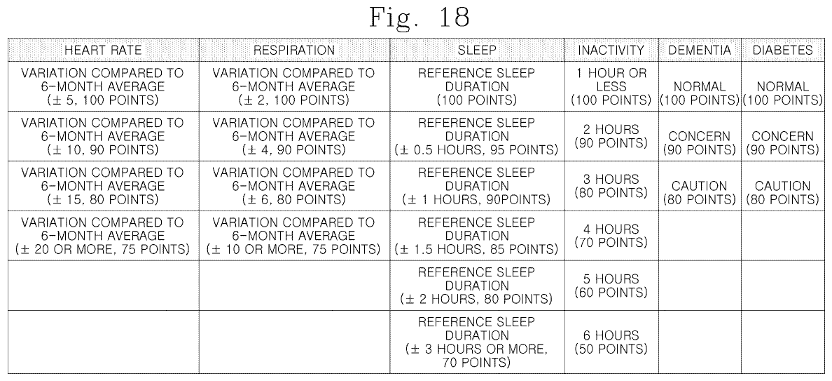

Further, according to an embodiment, the health analysis part 140 may provide a health score, which is a composite score obtained by evaluating the user's 1 health state so that changes in the user's 1 life pattern are easily recognized. Referring to FIG. 18, the health score may be set, on the basis of an individual score for each item, such as heart rate, respiration, sleep, inactivity, dementia, and diabetes, to be an average value thereof. However, no limitation thereto is imposed. The health score may be obtained in various ways, such as obtaining a weighted average by applying weightings corresponding to respective items. In addition, according to an embodiment, each user may drag items of high interest to him or her to change the order of the items. Herein, different weightings may be applied according to the order of the items.

For example, when user information is registered, “health interest” for each user may be set. With guidance text, such as “please select the items that you are most concerned about or interested in in order”, a user interface (UI) may be provided to enable a user to select items, such as heart rate, respiration, sleep, inactivity, dementia, and diabetes, in a desired order. Herein, when the user doesn't make his or her selection or selects “automatic”, weightings may be determined in the following order: heart rate, respiration, sleep, dementia, diabetes, and inactivity, which is the default setting. However, when a user has diabetes, the user may change the diabetes item to be first in order in the default setting. When a user has insomnia, the user may change the sleep duration item to be first in order in the default setting. In addition, when a user who is obese or has waist circumference issues, the user may determine the inactivity item to be first in order in the default setting. In addition, for a user having two or more chronic diseases, for example, a user having diabetes and insomnia may set the sleep duration item to be first in order and the diabetes item to be second in order, and may leave the rest to the default setting. Herein, the default setting is for illustrative purposes only, and the items or the order thereof included in the default setting may vary according to an embodiment.

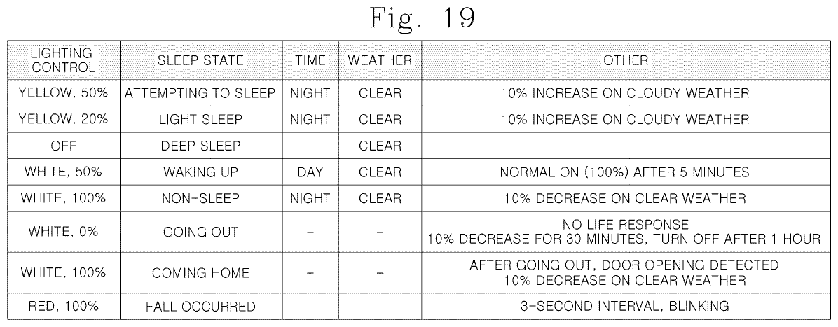

The environment controller 150 may control illuminance or color, heating and cooling, and ventilation in the home. Herein, the environment controller 150 may perform various controls in the home, considering the user's 1 types of sleep, life response, mood state, current time, weather, whether the user has gone out, and whether the user has fallen. That is, various IoT devices may be included in the home, so the environment controller 150 may automatically control the in-home environment in conjunction with the IoT devices. For example, as shown in FIG. 19, the environment controller 150 may control the lighting in various ways depending on weather, time, activity level, presence, and other conditions. Herein, the color may be changed in various ways according to the user's 1 preference, but the color and the brightness are functions provided by the light, and the environment controller 150 may apply the function only when a light providing the function is used. In the case of a light that does not provide the function, the environment controller 150 may provide only the on/off function for the lighting.

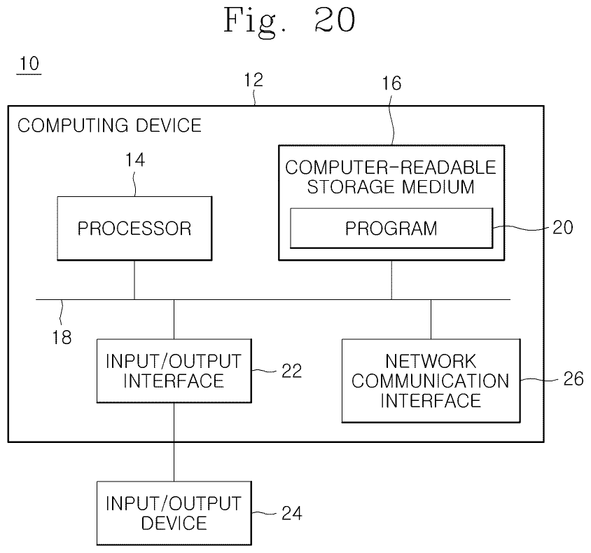

FIG. 20 is a block diagram illustrating a computing environment 10 suitable for use in exemplary embodiments. In the shown embodiment, each of the components may have different functions and capabilities other than those described below, and additional components may be included other than those described below.

The shown computing environment 10 includes a computing device 12. In an embodiment, the computing device 12 may be the health care monitoring and smart home convergence system 100 or the health analysis part 140 according to an embodiment of the present disclosure.

The computing device 12 may include at least one processor 14, a computer-readable storage medium 16, and a communication bus 18. The processor 14 may cause the computing device 12 to operate according to the above-mentioned exemplary embodiments. For example, the processor 14 may execute one or more programs stored in the computer-readable storage medium 16. The one or more programs may include one or more computer-executable instructions. When executed by the processor 14, the computer-executable instructions may be configured to cause the computing device 12 to perform operations according to the exemplary embodiments.

The computer-readable storage medium 16 is configured to store computer-executable instructions or program code, program data, and/or other suitable forms of information. The program 20 stored in the computer-readable storage medium 16 may include a set of instructions executable by the processor 14. In an embodiment, the computer-readable storage medium 16 may be a memory (volatile memory such as random-access memory, non-volatile memory, or any suitable combination thereof), one or more magnetic disk storage devices, optical disk storage devices, flash memory devices, any other form of storage medium accessible by the computing device 12 and capable of storing desired information, or any suitable combination thereof.

The communication bus 18 interconnects other various components of the computing device 12 including the processor 14 and the computer-readable storage medium 16.

The computing device 12 may also include one or more input/output interfaces 22 that provide interfaces for one or more input/output devices 24, and one or more network communication interfaces 26. The input/output interface 22 and the network communication interface 26 are connected to the communication bus 18. The input/output device 24 may be connected to other components of the computing device 12 through the input/output interface 22. Examples of the input/output device 24 may include input devices, such as pointing devices (a mouse or a trackpad), a keyboard, a touch input device (a touch pad or a touch screen), a voice or sound input device, various types of sensor devices and/or photographing devices; and/or output devices, such as a display device, a printer, a speaker, and/or a network card. An exemplary input/output device 24 may be included inside the computing device 12 as a component of the computing device 12, or may be connected to the computing device 12 as a separate device distinct from the computing device 12.

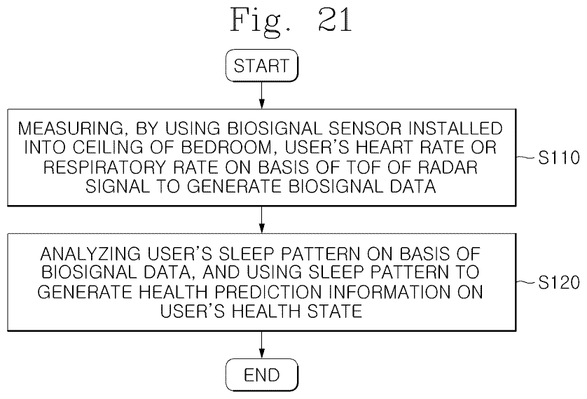

FIG. 21 is a flowchart illustrating a health monitoring method according to an embodiment of the present disclosure. Herein, each of the steps shown in FIG. 21 may be performed by a health care monitoring and smart home convergence system based on a built-in ceiling IoT radar sensor according to an embodiment of the present disclosure.

Referring to FIG. 21, the health care monitoring and smart home convergence system may measure the heart rate or the respiratory rate of a user on the basis of ToF of a radar signal to generate biosignal data by using an IoT radar sensor of a recessed type installed into the ceiling of a bedroom in a built-in manner in step S110. Herein, the IoT radar sensor may use ToF of a radar signal to measure a distance to a user, and may measure the heart rate and the respiratory rate of the user by detecting the movement of the user's chest on the basis of variations in the measured distance.

However, the measurement distance values measured by the IoT radar sensor may include noise due to a measurement environment, such as a bedroom with inconsistent temperature and humidity, and the user's movement. Therefore, in order to increase the accuracy of the respiratory rate and the heart rate measured by the IoT radar sensor, the health care monitoring and smart home convergence system may perform removal of noise. Specifically, the IoT radar sensor may obtain a measurement distance value to the user from the ToF of a reflection signal detected by each receiver, and may obtain distance variation, which is the difference between measurement distance values changed for a set time interval (for example, 1 msec). That is, the transmitter outputs radar signals at set time intervals, and the distance variation may be obtained by comparing the measurement distance value previously measured and with the measurement distance value currently measured. Afterward, it may be determined whether the current distance variation is noise by comparing the distance variation with the cumulative average value of the distance variations accumulated for each of the receivers. That is, when the distance variations obtained from the reflection signals received from the respective receivers are compared to the cumulative average value and within the set error range, the reflection signals may be regarded as signals reflected back from the actual user. However, when the distance variation obtained from the reflection signal is out of the set error range of the cumulative average value, the distance variation corresponds to noise and may be excluded. The biosignal data may be generated on the basis of the remaining distance variations excluding noise.

Afterward, the health care monitoring and smart home convergence system may analyze the user's sleep pattern on the basis of the biosignal data, and may use the sleep pattern to generate the health prediction information on the user's health state in step S120. Specifically, the health care monitoring and smart home convergence system may classify the types of sleep of the user by using the number-of-times variations in the respiratory rate or the heart rate of the user. For example, when the number-of-times variation in the respiratory rate or the heart rate is small, between 0˜ 5%, it may be determined that the user is in the deep sleep state. When the number-of-times variation in the respiratory rate or the heart rate is between 6˜ 35%, it may be determined that the user is in REM sleep. In addition, when the number-of-times variation in the respiratory rate or the heart rate is equal to or greater than 36%, it may be determined that the user is in a non-sleep state in which the user can not fall asleep and tosses and turns. Afterward, the health care monitoring and smart home convergence system may generate sleep data including the types of sleep occurring while the user is sleeping and the time corresponding to each of the types of sleep, and may collect the sleep data at regular time intervals to generate statistics. That is, the health care monitoring and smart home convergence system may analyze, on the basis of the sleep data, change trends for the total sleep duration, the deep sleep duration, the REM sleep duration, and the non-sleep duration to generate the health prediction information of the user. In this case, the health prediction information may attract the user's interest by providing the user with information on diseases that may occur depending on each sleep duration change, and may also provide the user with information on activities and the amount of required sleep to prevent the diseases.

Additionally, the health care monitoring and smart home convergence system may use the biosignal data to generate the resting heart rate (RHR) of the user, and may generate, on the basis of the resting heart rate, the health prediction information including the possibility of occurrence of various diseases (for example, Alzheimer's disease, Parkinson's disease, stroke, diabetes, and myocardial infarction) to the user. That is, the health care monitoring and smart home convergence system may provide the health prediction information that classifies, on the basis of the change trends in the resting heart rate, the possibility of occurrence of diabetes or dementia, into “normal”, “concern”, and “caution”. Herein, the health care monitoring and smart home convergence system may increase the reliability of the possibility of occurrence of each disease, further considering the user's 1 waist circumference, blood pressure measurement value, and the change trends for the sleep duration. The user's waist circumference or blood pressure measurement value may be separately input from the user. Further, according to an embodiment, the health care monitoring and smart home convergence system may provide a health score, which is a composite score obtained by evaluating the user's health state so that changes in the user's life pattern are easily recognized.

Herein, although not shown, the health care monitoring and smart home convergence system may further include a step of detecting the user's positioning on a bed by using a positioning detection sensor that is installed within the bedroom and remotely measures the user's positioning to generate positioning data. The positioning detection sensor may generate the positioning data representing the user's positioning. When the positioning data corresponds to positioning other than sleep positioning within a preset estimated sleep duration, the health care monitoring and smart home convergence system may determine that the user has left and may process, as noise, the biosignal data measured while the user has left.

In addition, although not shown, the health care monitoring and smart home convergence system may further include a step of detecting the user's fall by using a fall detection sensor that is installed in the home and detects a fall that occurs to the user while walking in a set area. In the health care monitoring and smart home convergence system, when the positioning change speed of an object recognized by the fall detection sensor is equal to or greater than a set value and the central axis of the object, which is in the shape of a column of which the central axis is perpendicular to the ground, makes positioning change horizontal to the ground and remains in changed positioning for a set period of time or longer, it may be detected that a fall has occurred.

Further, the health care monitoring and smart home convergence system may control illuminance or color, heating and cooling, and ventilation in the home. That is, the health care monitoring and smart home convergence system may perform various controls in the home, considering the user's types of sleep, life response, mood state, current time, weather, whether the user has gone out, and whether the user has fallen. Various IoT devices may be included in the home, so the health care monitoring and smart home convergence system may automatically control the in-home environment in conjunction with the IoT devices.

The present disclosure described above may be implemented as computer-readable code on a medium on which a program is recorded. A computer-readable medium may continuously store computer-executable programs, or may temporarily store the same for execution or downloading. In addition, the medium may be various recording means or storage means in the form of a single hardware element or a combination of several hardware elements. The medium is not limited to a medium directly connected to any computer system, and may be distributed over a network. Examples of the medium include magnetic media, such as hard disks, floppy disks, and magnetic tapes; optical recording media, such as CD-ROMs and DVDs; magneto-optical media, such as floptical disks; and ROM, RAM, and flash memory, which are configured to store program instructions. In addition, other examples of the medium may include recording media or storage media managed by app stores that distribute applications, by sites that provide or distribute other various software elements, or by servers. Accordingly, the above detailed description should not be construed as restrictive in all respects but should be considered illustrative. The scope of the present disclosure should be determined by a reasonable interpretation of the appended claims, and all changes within the equivalent scope of the present disclosure are included in the scope of the present disclosure.

The present disclosure is not limited to the above-described embodiments and the accompanying drawings. It will be apparent to those skilled in the art that elements according to the present disclosure may be substituted, modified, and changed within the scope of the present disclosure.

What is claimed is:

1. A health care monitoring and smart home convergence system based on a built-in ceiling IoT radar sensor, the health care monitoring and smart home convergence system comprising:
the built-in ceiling IoT radar sensor installed into a ceiling of a bedroom, and configured to measure a user's heart rate or respiratory rate on the basis of time of flight (ToF) of a radar signal to generate biosignal data; and
a health analysis part configured to analyze the user's sleep pattern on the basis of the biosignal data, and use the sleep pattern to generate health prediction information on the user's health state.

2. The health care monitoring and smart home convergence system of claim 1, wherein the IoT radar sensor is installed semi-recessed by a fixing frame and a spring clip inside an installation hole formed in the ceiling of the bedroom in a built-in manner,
the fixing frame is inserted into the installation hole to support a lower portion of the IoT radar sensor and has an opening through which the IoT radar sensor is exposed, and
the spring clip is positioned between the installation hole and the fixing frame, and is configured to fix the fixing frame within the installation hole with elasticity of the spring clip.

3. The health care monitoring and smart home convergence system of claim 2, wherein the IoT radar sensor is powered by being coupled to a wire harness connected to the installation hole from the inside of the ceiling.

4. The health care monitoring and smart home convergence system of claim 1, wherein the IoT radar sensor comprises:
a transmitter configured to output the radar signal at set time intervals, and a receiver configured to receive a reflection signal obtained as the radar signal is reflected, wherein a ratio of the number of the transmitter to the number of the receiver included in the IoT radar sensor is 1:N (herein, N is an integer of 1 or greater), and a plurality of the receivers are positioned distributed in a preset area within the IoT radar sensor.

5. The health care monitoring and smart home convergence system of claim 4, wherein the IoT radar sensor is configured to obtain a measurement distance value to the user from the ToF of the reflection signal detected by each of the receivers, and obtain distance variation, which is a difference between the measurement distance values changed for the set time interval, and then compare the distance variation to a cumulative average value of the distance variations accumulated for each of the receivers to determine whether the distance variation is noise.

6. The health care monitoring and smart home convergence system of claim 5, wherein the IoT radar sensor is configured to determine that the distance variation is the noise when the distance variation is out of a set error range of the cumulative average value, and generate the biosignal data on the basis of the remaining distance variations excluding the noise.

7. The health care monitoring and smart home convergence system of claim 5, wherein the IoT radar sensor is configured to detect micro-movement of the user's chest from the distance variation to measure the user's heart rate or respiratory rate.

8. The health care monitoring and smart home convergence system of claim 1, wherein the health analysis part further comprises a sleep analysis part configured to analyze the user's sleep pattern on the basis of the biosignal data,
wherein the sleep analysis part is configured to classify types of sleep of the user according to number-of-times variations in the respiratory rate or the heart rate.

9. The health care monitoring and smart home convergence system of claim 8, wherein the sleep analysis part is configured to classify the types of sleep into at least one selected from a group of deep sleep, rapid eye movement (REM) sleep, and non-sleep, on the basis of a change range of the number-of-times variations.

10. The health care monitoring and smart home convergence system of claim 9, wherein the health analysis part is configured to generate the health prediction information on the basis of a sleep duration variation for at least one selected from a group of the user's total sleep duration, deep sleep duration, REM sleep duration, and non-sleep duration.

11. The health care monitoring and smart home convergence system of claim 8, wherein the sleep analysis part is configured to determine that the user has left when it is measured that the biosignal data is less than a limit value for a set period of time or longer, and process, as noise, the biosignal data measured while the user has left.

12. The health care monitoring and smart home convergence system of claim 8, further comprising:
a positioning detection sensor installed in the bedroom, and configured to remotely measure the user's positioning to generate positioning data,
wherein the sleep analysis part is configured to determine that the user has left when the positioning data corresponds to positioning other than sleep positioning within a preset sleep duration, and process, as noise, the biosignal data measured while the user has left.

13. The health care monitoring and smart home convergence system of claim 1, further comprising:
a fall detection sensor installed in the user's home, and configured to detect a fall that occurs to the user while walking in a set area,
wherein the health analysis part is configured to detect that the fall has occurred when a positioning change speed of an object recognized by the fall detection sensor is equal to or greater than a set value and a central axis of the object in the shape of a column of which the central axis is perpendicular to the ground makes positioning change horizontal to the ground and remains in changed positioning for a set period of time or longer.

14. The health care monitoring and smart home convergence system of claim 1, further comprising:
an environment controller configured to control color, illuminance, and on/off operation of lighting in the user's home depending on at least one selected from a group of the user's types of sleep, life response, current time, weather, whether the user has gone out, and whether the user has fallen.

15. The health care monitoring and smart home convergence system of claim 1, wherein the health analysis part is configured to use the biosignal data to generate the user's resting heart rate (RHR), and generate, on the basis of the resting heart rate, the health prediction information including possibility of occurrence of at least one disease among Alzheimer's disease, Parkinson's disease, stroke, diabetes, and myocardial infarction to the user.

16. The health care monitoring and smart home convergence system of claim 15, wherein the health analysis part is configured to receive, from the user, the user's waist circumference or blood pressure measurement value or both, and generate the health prediction information further including the received waist circumference or blood pressure measurement value.

17. The health care monitoring and smart home convergence system of claim 15, wherein the health analysis part is configured to generate a health score by comparing a reference value with at least one selected from a group of the user's heart rate, respiratory rate, sleep duration, and inactivity time, and provide the health score by including the health score in the health prediction information.

18. The health care monitoring and smart home convergence system of claim 1, wherein the health analysis part is configured to transmit a notification message including the health prediction information to a wall pad installed in the user's home or a pre-registered user terminal.

19. A health monitoring method using a health care monitoring and smart home convergence system based on a built-in ceiling IoT radar sensor installed in a home, the health monitoring method comprising:
measuring, by using the built-in ceiling IoT radar sensor installed into a ceiling of a bedroom, a user's heart rate or respiratory rate on the basis of time of flight (ToF) of a radar signal to generate biosignal data; and
analyzing the user's sleep pattern on the basis of the biosignal data, and using the sleep pattern to generate health prediction information on the user's health state.

20. A computer program stored on a medium, in combination with hardware, to perform a health monitoring method of claim 19.
